(19) United States
(12) Patent Application Publication
(10) Pub. No.: US 2018/0085289 A1
(43) Pub. Date: Mar. 29, 2018

(54) COSMETIC COMPOSITION FOR MIMICKING MOISTURIZED GLOW

(71) Applicant: AMOREPACIFIC CORPORATION, Seoul (KR)

(72) Inventors: Chun Bok JUNG, Yongin-si, Gyeonggi-do (KR); Se Jun PARK, Yongin-si, Gyeonggi-do (KR); Yu Jin KANG, Yongin-si, Gyeonggi-do (KR); Kyung Ho CHOI, Yongin-si, Gyeonggi-do (KR); Young Jin CHOI, Yongin-si, Gyeonggi-do (KR)

(73) Assignee: AMOREPACIFIC CORPORATION, Seoul (KR)

(21) Appl. No.: 15/563,191

(22) PCT Filed: Mar. 31, 2016
(86) PCT No.: PCT/KR2016/003312

§ 371 Date: Sep. 29, 2017

(30) Foreign Application Priority Data
Mar. 31, 2015 (KR) 10-2015-0045699

(51) Int. Cl. A61K 8/02; A61K 8/26; A61K 8/25; A61K 8/29; A61K 8/19; A61Q 1/12; A61Q 1/02

(52) U.S. Cl. CPC A61K 8/0258; A61K 8/022; A61K 8/26; A61Q 1/02; A61K 8/29; A61K 8/19; A61Q 1/12; A61K 8/25

(57) ABSTRACT

The present invention relates to a cosmetic composition for mimicking a moisturized glow, and specifically, to a composition that can mimic a moisturized glow by containing a specific combination of composite powders having various colors.

### TECHNICAL FIELD

The present invention relates to a cosmetic composition for mimicking a moisturized glow. More particularly, the present invention relates to a composition for mimicking a moisturized glow by containing a specific combination of composite powders having various colors.

### BACKGROUND ART

The realization of ideal skin through cosmetics has become an important concern of customers. In order to satisfy these needs of customers, studies are performed in the art in various manners, for example, by mimicking an ideal skin tone through skin color studies for each region or by correcting a skin tone through the expression of colors at a specific wavelength. One approach to implementing these methods is to realize a moisturized glow on a skin.

Studies associated with a skin change caused by moisture have been performed mainly as index such as moistness, trans-epidermal water loss (TEWL) in skin, or the like. However, the optical properties associated with moisture have only been studied extensively, for example, a skin applied with moisturizer looking darker than before application of the moisturizer, optical patterns being varied depending on the amount of moisture in vitro, or the like. In general, studies for understanding the exact optical properties shown by moisture and implementing the same are still not satisfactory.

In a color matching system of a makeup formulation that has been commonly used up to date, a method of expressing a color by adding yellow, red, and black pigments to a white base has been utilized. However, this method was not sufficient to accurately mimic a phenomenon where a particular color is highlighted while getting bright, which is an alteration in a spectrum positive (+) direction. As an approach to solve these problems, a finely divided pearl powder was used.

However, when a conventional pearl powder is applied to a cosmetic composition, there have been disadvantages that the applicability, uniformity and formability deteriorate due to powders having various sizes, and the interference effect does not occur uniformly, resulting in shiny appearance. In order to supplement these disadvantages, attempts have been made to reduce the particle size of pearl powder and narrow the particle distribution, but there remains still a disadvantage that a cloudy phenomenon occurs.

### PRIOR ART DOCUMENT

### Patent Document

Korean Patent Application Publication No. 10-2013-0060583 (published on Jun. 10, 2013)

### DETAILED DESCRIPTION OF THE INVENTION

### Technical Problem

Thus, the present inventors have attempted to provide an accurate physical interpretation of the term “moisturized glow” widely used in the art in connection with skin expression and also to provide a combination of composite powders which can mimic such moisturized glow.

Therefore, the object of the present invention is to provide a cosmetic composition comprising a combination of composite powders having various colors, capable of making the skin tone uniform and glossy while concealing defects of skin due to interference effects.

### Technical Solution

In order to achieve the above objects, the present invention provides a cosmetic composition comprising a composite powder mixture composed of three or more composite powders selected from the group consisting of composite powders of purple color, red color, bronze color, red copper color, reddish brown color, indigo color, blue color, green color, yellow color and gold color, wherein said composite powder is a plate-like fine powder coated with an inorganic material.

In addition, the present invention provides use of a composite powder as an agent for mimicking a moisturized glow in the preparation of a cosmetic composition.

### Advantageous Effects

The composition of the present invention can mimic a moisturized glow due to a scattering phenomenon of ligh by using an appropriate combination of composite powders having various colors, thereby efficiently correcting the skin tone while concealing skin wrinkles and dullness.

### BRIEF DESCRIPTION OF DRAWINGS

FIG. 1 shows the patterns extracted by analyzing the entire range of visible light spectrum at the skin of the moisture-supersaturated state. Here, p-comb represents a composition (Example 1) comprising composite powders, the horizontal axis represents spectrum, and the vertical axis represents values at the spectrum. As to the numbers on the horizontal axis, the range of 360 nm to 370 nm is assigned to No. 1 section, and from this, numbering is made with an interval of 10 nm.

### DETAILED DESCRIPTION OF THE EMBODIMENTS

The present invention provides a cosmetic composition capable of mimicking a moisturized glow. It has been found that by applying the cosmetic composition of the present invention, a skin image which is very similar to a skin state under a moisture-supersaturated condition can be provided even without being separately supplied with any moisture or applied with any moisturizing product, and there is no significant difference in the result.

The term “moisturized glow” as used herein refers to an optical property that moisture represents, and refers to an aspect of patterned spectrum change in a moisture oversaturation state. In particular, in a moisture-supersaturated state of a skin, a significant pattern change of spectrum is observed in the range of purple color to orange color, which refers to “moisturized glow”. In particular, the spectrum wavelength range of visible light in a moisture oversaturation condition or in a state of mimicking a moisturized glow is in the range of 360 nm to 740 nm, more preferably 380 nm to 630 nm, still more preferably 360 nm to 550 nm.

The cosmetic composition of the present invention comprises a composite powder which is a plate-like fine powder coated with an inorganic material. Further, the plate-like fine composite powder coated with an inorganic material may be optionally subjected to coating treatment with amorphous silica, silicone or dimethicone, or the like.

The plate-like fine powder used in the composition of the present invention is one or more selected from a group consisting of mica, synthetic mica, alumina, borosilicate powder, talc and sericite, and preferably synthetic mica.

Further, the inorganic material used for coating the fine powder in the present invention is one or more selected from the group consisting of titanium dioxide, iron oxide and tin oxide, and preferably titanium dioxide.

The composite powder used in the present invention has an average particle size ranging from 2 to 16 μm. When the particle size of the composite powder falls within the above range, it can be used like a coloring matter without giving any pearl-like feeling when observed by the naked eye.

The composite powder used in the present invention is composed of three or more composite powders selected from the group consisting of composite powders of purple color, red color, bronze color, red copper color, reddish brown color, indigo color, blue color, green color, yellow color and gold color, and these composite powders exhibit different colors depending on the thickness of a coated layer of the inorganic material covered on a fine powder. This is already known in the art and variously applied as inorganic powders exhibiting an interference effect of light in all cosmetic compositions.

The composition of the present invention uses a composite powder mixture composed of three or more composite powders selected from the group consisting of composite powders of purple color, red color, bronze color, red copper color, reddish brown color, indigo color, blue color, green color, yellow color and gold color. Preferably, the composite powders of purple color, blue color, green color and yellow color are mixed and used. In this case, it is preferable to mix and use the composite powders of purple color, blue color, green color and yellow color in a weight ratio of 5˜9:4.5˜8.5:11˜15:2˜6, particularly in a weight ratio of 6.5˜7.5:6˜7:12.5˜13.5:3.5˜4.5.

The methods for producing a composite powder coated with an inorganic material according to the present invention are not particularly limited, and examples thereof are as follows.

First, composite powders of colors to be mixed, specifically three or more composite powders selected among composite powders of purple color, red color, bronze color, red copper color, reddish brown color, indigo color, blue color, green color, yellow color and gold color are prepared. These composite powders are commercially available or can be directly prepared by coating a fine powder with an inorganic material (for example, titanium dioxide) in a desirable thickness (here, the color of composite powder is decided depending on the thickness of the coating material and the coating thickness generally falls within a range of 1 nm to 100 μm, preferably 10 nm to 20 μm). And the above composite powders are introduced into a Henschel mixer and slowly mixed at RPM 500 or below. The mixing time is preferably 60 to 300 seconds. The particle size of the composite powders thus prepared is measured and a certain amount of sample is taken. The color is confirmed via a colorimeter, and then the process is finished.

The cosmetic composition of the present invention comprises the coated composite powders, thereby imparting optical features similar to a state where skin is supersaturated with moisture, and also mimicking a moisturized glow by applying the composition to a skin. Further, a soft focus effect can be provided, thus immediately supplementing skin defects and making skin condition better visible.

The cosmetic composition according to the present invention comprises a mixture of the coated composite powders in an amount of 0.001 to 30% by weight, preferably 0.01 to 20% by weight, based on the total weight of the composition. In case of a powder formulation, for example, it may be applied in a large amount of 10 to 20% by weight, but in case of formulations other than powder, it is more preferable to be used in an amount of 0.01 to 5% by weight. The reason is because, when used in an amount of 5% by weight or less, delta E would be measured at a value of 1.5 or less, and thus, it would not actually feel a big difference from an excess moisture state when observed with naked eye.

The type of the cosmetic composition is not particularly limited, but the cosmetic composition can be variously applied to base makeup cosmetics such as powder fact, two way cake, face powder, foundation, makeup primer, makeup base, BB cream, or the like; skin care cosmetics such as cream, lotion, emulsion; color cosmetics such as eye shadow, lipstick, blusher, or the like.

Further, when the formulation of the cosmetic composition according to the present invention is in the form of powder or spray, lactose, talc, silica, aluminum hydroxide, calcium silicate or polyamide powder may be used as a carrier component, and particularly in the form of spray, a propellant such as chlorofluorohydrocarbon, propane/butane or dimethyl ether may be additionally included.

When the formulation of the cosmetic composition according to the present invention is in the form of solution or emulsion, a solvent, a solvating agent or an emulsifying agent may be used as the carrier component, and examples thereof include water, ethanol, isopropanol, ethyl carbonate, ethyl acetate, benzyl alcohol, benzyl benzoate, propylene glycol, 1,3-butylglycol oil, glycerol aliphatic ester, polyethylene glycol or sorbitan fatty acid ester.

When the formulation of the cosmetic composition according to the present invention is in the form of suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tracanth or the like may be used as the carrier component.

The cosmetic composition may further include, in addition to the above coated composite powders, functional additives and components that may be contained in a conventional cosmetic composition. The above functional additive may include a component selected from the group consisting of a water-soluble vitamin, an oil-soluble vitamin, a high-molecular peptide, a high-molecular polysaccharide, a sphingolipid and a seaweed extract.

In the cosmetic composition of the present invention, components contained in a general cosmetic composition may be blended in addition to the above-mentioned functional additives, if necessary. A blending component which may be added herein may include a fat component, a moisturizing agent, an emollient, a surfactant, organic and inorganic pigments, an organic powder, a UV absorber, an antiseptic, a bactericide, an antioxidant, an herbal extract, a pH regulating agent, an alcohol, a coloring matter, an aromatic, a blood flow stimulant, a cooling agent, an antiperspirant, purified water, etc.

Hereinafter, the present invention will be described in detail by way of examples. However, the examples are only for illustrative purposes and not intended to limit the scope of this disclosure thereto.

### [Reference Example] Preparation of a Composition Containing Coated Composite Powders (Example 1)

A skin care composition containing composite powders of purple color, blue color, green color and yellow color (Sharon Soft focus; CQV Co. Ltd., located in South Korea) was prepared in the composition shown in the following Table 1 in accordance with a conventional production method.

TABLE 1

| Component | Content (Unit: % by weight) |
|---|---|
| Deionized water | To 100 |
| EDTA-2NA | 0.05 |
| Glycerin (Cosmetic Material Standard, | 5 |
| KCID) | |
| Sepinov EMT10 | 0.6 |
| 1,3-Butylene glycol (SK Grade) | 5.5 |
| TWEEN #20 | 0.6 |
| DC345 | 4 |
| KSG-16 | 2 |
| SH200C-6CS | 4 |
| TWEEN #80 | 0.1 |
| DC2-1184 | 2 |
| Sharon Soft focus Violet | 1.15 |
| Sharon Soft focus Blue | 1.06 |
| Sharon Soft focus Green | 2.14 |
| Sharon Soft focus Yellow | 0.66 |
| Sepiplus 400 | 0.3 |

### [Test Example 1] Analysis of the Spectrum of Moisture and Moisturized Glow

Onto faces of 10 subjects who had various skin colors, a pack impregnated only with water (a mask sheets made with a conventional nonwoven fabric and then wetted with water) is applied only to the right side of a face to cause a moisture-supersaturated state, the composition of Example 1 was applied to the left side of the face, and the spectrum was measured after 15 minutes. At this time, in order to maximally verify whether there is any difference depending on a variety of skin colors and skin sites, measurements have been performed on the cheek and forehead skins at the same time. The measurement results are shown in Table 2 below.

TABLE 2

| Significant | Verified value of the difference of before/after | | |
|---|---|---|---|
| difference | L | a | b |
| Moisture | 0.000158 | 0.061273 | 0.993062 |

Values of 0.05 or less being accepted as having a significant difference
N=18

As shown in Table 2, it could be confirmed that the color of skin in vivo became bright when moisture became supersaturated, and such characteristics can be demonstrated by the fact that a significant change was induced at only L value among L, a, b, color system.

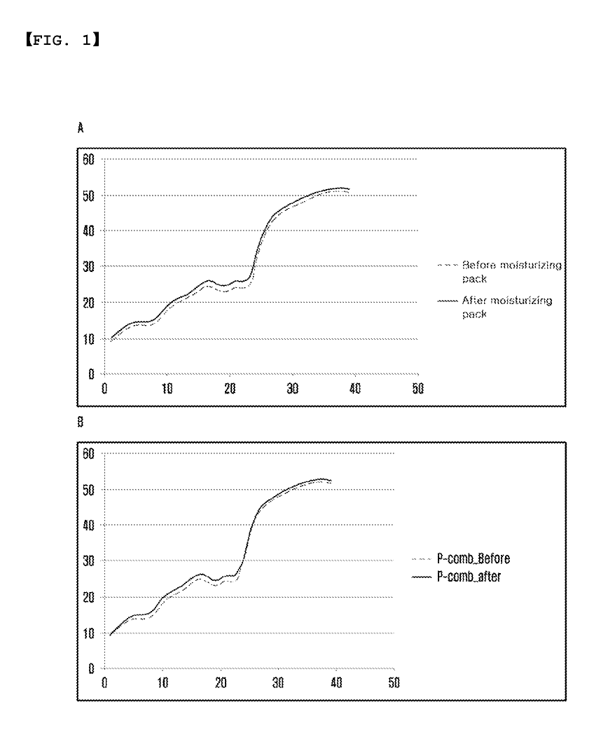

Further, it could be confirmed from the above measurements that optical properties of skin in a moisture oversaturation state are phenomena generally observed regardless of the skin colors and the face sites. However, because of the lack of information for the purpose of mimicking the features representing a simple brightness change, the entire spectrum of visible light is analyzed to extract a pattern. The results obtained are shown in FIG. 1.

As a result of observing the spectrum changes, a significant patterned change was observed in the range of purple to yellow as defined as a moisturized glow in the present invention, and it could be confirmed that the skin in a moisture-supersaturated state and the skin to which the composition of Example 1 prepared in accordance with the present invention has been applied exhibited almost the same aspect of spectrum change (the section of spectrum wavelength of 630 nm or more, corresponding to the section of No. 28 or higher of the spectrum can be ignored. The section showing slight differences in the graph can be ignored since it corresponds to the section of No. 28 or higher and is not a significant interval representing a spectrum change aspect).

In addition, it could be observed that, by applying the composite powder mixture according to the present invention, the light quantity increases in a very similar manner as the moisture oversaturation state, and so it could be confirmed that the correction effect of a skin tone could be obtained therefrom.

1. A cosmetic composition comprising, as effective constituents, a composite powder mixture composed of three or more of composite powders selected from the group consisting of composite powders of purple color, red color, bronze color, red copper color, reddish brown color, indigo color, blue color, green color, yellow color and gold color, wherein said composite powders are plate-like fine powders coated with an inorganic material.

2. The cosmetic composition according to claim 1, wherein said composite powder mixture is a mixture of composite powders of purple color, blue color, green color and yellow color.

3. The cosmetic composition according to claim 2, wherein said composite powder mixture has a weight ratio of 5˜9:4.5˜8.5:11˜15:2˜6 of composite powder of purple color:composite powder of blue color:composite powder of green color:composite powder of yellow color.

4. The cosmetic composition according to claim 1, wherein said composite powder mixture has a spectrum wavelength range of visible light of 360 nm to 740 nm.

5. The cosmetic composition according to claim 1, wherein said composite powder mixture has a spectrum wavelength range of visible light of 380 nm to 630 nm.

6. The cosmetic composition according to claim 1, wherein said powder is one or more selected from a group consisting of mica, synthetic mica, alumina, borosilicate powder, talc, and sericite.

7. The cosmetic composition according to claim 1, wherein said inorganic material is one or more selected from a group consisting of titanium dioxide, iron oxide and tin oxide.

8. The cosmetic composition according to claim 1, wherein said composite powder has an average particle size of 2 to 16 μm.

9. The cosmetic composition according to claim 1, wherein the composition comprises a composite powders mixture in an amount of 0.001 to 30% by weight.

10. The cosmetic composition of claim 1, wherein said composition is formulated in the form of a base makeup cosmetic, a skin care cosmetic or a color cosmetic.

11. The cosmetic composition as claimed in claim 1, wherein said composition mimics a moisturized glow.

12. Use of composite powders as an agent for mimicking a moisturized glow, in the preparation of a cosmetic composition.
